(19) United States
(12) Patent Application Publication
(10) Pub. No.: US 2025/0009734 A1
(43) Pub. Date: Jan. 9, 2025

(54) METHODS OF INHIBITING FOXO1 FOR TREATMENT OF LYMPHATIC DISORDERS

(71) Applicant: UNIVERSITY OF SOUTH FLORIDA, Tampa, FL (US)

(72) Inventor: Ying Yang, Tampa, FL (US)

(21) Appl. No.: 18/757,228

(22) Filed: Jun. 27, 2024

Related U.S. Application Data
(60) Provisional application No. 63/510,587, filed on Jun. 27, 2023.

(51) Int. Cl. A61K 31/47

(52) U.S. Cl. CPC A61K 31/47

(57) ABSTRACT

Disclosed are methods for treating congenital lymphedema or increasing lymphatic valve growth by administering a FOXO1 inhibitor. Also disclosed are methods for increasing nuclear β-catenin activity in a cell by contacting the cell with a FOXO1 inhibitor.

### CROSS-REFERENCE TO RELATED APPLICATIONS

This application claims the benefit of and priority to U.S. Provisional Application No. 63/510,587 filed on Jun. 27, 2023, the content of which is incorporated by reference in its entirety.

### STATEMENT REGARDING FEDERALLY SPONSORED RESEARCH

This invention was made with government support under HL145397 awarded by the National Heart, Lung, and Blood Institute. The government has certain rights in the invention.

### BACKGROUND

The lymphatic vasculature is essential for the absorption of interstitial fluids to form lymph and for the transportation of lymph back to the blood vasculature. The lymphatic capillaries absorb interstitial fluids and the collecting lymphatic vessels transport lymph (Yang and Oliver, 2014, Breslin et al., 2018). To efficiently transport lymph against the hydrostatic pressure gradient, the collecting lymphatic vessels pump to move lymph forward through perivascular smooth muscle cell contraction. Lymphatic valves in the collecting lymphatic vessels act as gates and open when the pressure builds up downstream of the valve leaflets to let lymph pass. Valves close when the upstream pressure of the valve leaflets is higher than the downstream pressure to ensure the forward movement of lymph in one direction (Davis et al., 2011). This unidirectional lymph flow is important for the function of the lymphatic vasculature, which is maintained by lymphatic valves. Retrograde lymph flow is one of the diagnostic characters for the lymphatic disease, lymphedema.

Mutations in many valve genes have been identified to be a causative factor for congenital lymphedema in human patients and the corresponding knockout mouse models of these genes have shown defects in lymphatic valve formation. For example, Heterozygosity of FOXC2 gene causes lymphedema-distichiasis syndrome in human patients (Kriederman et al., 2003). Foxc2 is a forkhead box, winged-helix transcription factor and regulates the maturation of collecting lymphatic vessels. During embryonic development, no lymphatic valves form in Foxc2 null mice (Norrmen et al., 2009). Moreover, Foxc2 is crucial for maintaining valves throughout life. Postnatal deletion of Foxc2 results in loss of lymphatic valves after the valve structure has formed (Sabine et al., 2015). In the Foxc2 heterozygous mouse model, the number of lymphatic valves is reduced by nearly 50% in the skin and the mesentery (Kanady et al., 2015, Scallan et al., 2021). The function of the remaining valves in Foxc2 heterozygous mice is severely compromised (Scallan et al., 2021, Castorena-Gonzalez et al., 2020). Therefore, the Foxc2 heterozygous mice are a model for human autosomal dominant lymphedema-distichiasis syndrome demonstrating the defects in lymphatic valves.

The formation of lymphatic valves requires oscillatory shear stress (OSS) from lymph flow. The adherens junction protein VE-cadherin on the cell membrane functions as a key component in the mechanotransduction signaling pathway to transmit the external OSS signal into the lymphatic endothelial cells (LECs) through cytoplasmic AKT activation (Yang et al., 2019). The OSS signaling upregulates the expression of three key valve transcription factors Prox1, Foxc2, and Gata2 to initiate the specification of lymphatic valve cells (Sweet et al., 2015). Like FOXC2, GATA2 is required for lymphatic valve formation and maintenance (Kazenwadel et al., 2015). Mutations in GATA2 causes primary lymphedema in human patients (Kazenwadel et al., 2012, Ostergaard et al., 2011). The activation of these valve-forming genes is regulated by the transcription factors, β-catenin and Foxo1, which are downstream targets in the OSS-AKT signaling pathway. β-catenin binds to the promoter regions of PROX1 and FOXC2, which activates the expression of these genes. Loss of β-catenin in LECs leads to defects in valve formation during embryonic development (Cha et al., 2016). In contrast, Foxo1 is a repressor for lymphatic valve formation. When FOXO1 is in the nucleus, it binds to the promoter area of FOXC2 to repress its expression (Scallan et al., 2021). However, when activated AKT phosphorylates FOXO1, FOXO1 no longer stays in the nucleus and is degraded in the cytoplasm. Therefore, deletion of Foxo1 in the lymphatics results in increased valve formation in healthy mice. Moreover, Foxo1 ablation in Foxc2 heterozygous mice, the model of human lymphedema-distichiasis disease, rescues both the valve loss and valve dysfunction to the wildtype level (Scallan et al., 2021). Therefore, pharmacological inhibition of FOXO1 should be explored as a viable strategy to rescue valve defects in congenital lymphedema.

### SUMMARY

In an aspect, a method for treating congenital lymphedema in a subject in need thereof is provided, the method comprising administering to the subject a therapeutically effective amount of a FOXO1 inhibitor. In embodiments, the subject has lymphedema-distichiasis syndrome. The subject may be between about 0 and about 60 months old.

The FOXO1 inhibitor may be administered intraperitoneally. The FOXO1 inhibitor may be AS1842856. The AS1842856 may be administered at a dose of between about 0.05 mg/kg and about 10 mg/kg. In embodiments, the AS1842856 is administered at a dose of about 8 mg/kg. The AS1842856 may be administered at about every other day.

The subject may be a human or a non-human mammal.

In another aspect, a method for increasing lymphatic valve growth in a subject in need thereof is provided, the method comprising administering to the subject a FOXO1 inhibitor. The FOXO1 inhibitor may be administered intraperitoneally. The FOXO1 inhibitor may be AS1842856. The subject may be a human or a non-human mammal.

In another aspect, a method for increasing nuclear β-catenin activity in a cell is provided, the method comprising contacting the cell with a FOXO1 inhibitor. The cell may be a lymphatic endothelial cell. In embodiments, the cell is a human dermal lymphatic endothelial cell.

The FOXO1 inhibitor may be AS1842856. The cell may be contacted with the AS1842856 for about 48 hours. The cell may be contacted with the AS1842856 at a concentration of between about 0.1 μM and about 1 μM. In embodiments, the cell is contacted with the AS1842856 at a concentration of about 0.5 μM.

### BRIEF DESCRIPTION OF THE DRAWINGS

The patent or patent application file contains at least one drawing in color. Copies of this patent or patent application publication with color drawing(s) will be provided by the Office upon request and payment of the necessary fee.

Non-limiting embodiments will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. AS1842856 (AS) has a time and concentration dependent effect on valve genes expression. Human dermal lymphatic endothelial cells (hdLECs) were cultured under static conditions and treated for the indicated time (24, 48, and 72 hours) and concentrations from left to right: DMSO 1 μM, AS 0.1 μM, AS 0.5 μM, and AS 1 μM. Afterwards, mRNA of indicated genes was extracted, reverse transcribe and quantified by qRT-PCR analysis. All values are mean±SEM. n=3 experiment repeats; ns=not significant; *p≤0.05, **p≤0.01.

FIGS. 2A-2F. AS1842856 elevates protein expression of valve genes and promotes b-catenin nuclear localization in cultured hdLECs. (A) hdLECs were cultured under static condition and treated for 48 hours with 0.5 μM DMSO (vehicle control) or 0.5 μM AS treatment. Thereafter, proteins were extracted, and western blot perform for the indicated proteins. ACTB is the loading control. (B) Quantification results of indicated proteins in western blot from three independent experiments. (C-F) Immunostaining of cultured hdLECs for PROX1 and CTNNB1 protein. All values are mean±SEM. n=3 experiment repeats; ns=not significant; *p≤0.05, **p≤0.01, ***p≤0.001. Vehicle control: 0.5 μM DMSO for 48 hours treatment. AS1842856: 0.5 μM AS1842856 for 48 hours treatment. Cells were stained for PROX1 (green) and CTNNB1 (purple). (E) and (F) are the merged images for PROX1 and CTNNB1. Scale bar is 50 μm.

FIGS. 3A-3F. AS1842856 treatment in vivo increases valve number in Foxc2^{+/CreERT2} mice. (A) Schematic illustration of AS1842856 injection procedure in postnatal day (P) 7-21 pups. (B-E) Fluorescent images showing the morphology of mesenteric collecting lymphatic vessels at P21. Mesenteries are from Prox1-GFP with DMSO (vehicle control) injection (B); Prox1-GFP with AS1842856 injection (C); Foxc2^{+/CreERT2}; Prox1-GFP with DMSO injection (D); Foxc2^{+/CreERT2}; Prox1-GFP with AS1842856 injection (E). (F) Quantification of valves per millimeter at P21 from 4 groups of mice. 4 mice are used for the analysis. All values are mean±SEM. Two-way ANOVA was performed with Turkey's multiple comparisons. ns=not significant, *p≤0.05, ***p<0.001. Scale bar is 1000 μm.

FIG. 4. AS1842856 injection promotes b-catenin nuclear localization in vivo. Panels A-P′ show whole-mount immunostaining of P10 mesenteric collecting lymphatic vessels from wildtype and Foxc2^{+/CreERT2} pups with vehicle control (DMSO) or AS1842856 injection. Prox1 in green, Ctnnb1 in purple, and VE-cadherin in gray. A′-P′ are the higher magnification images of the white box areas in A-P. Scale bars are 100 μm.

FIGS. 5A-5I. Overactivation of β-catenin in nucleus rescues valve loss in Foxc2^{+/CreERT2} mice. (A) Schematic illustration of tamoxifen injection procedure that results in deletion of exon 3 of Ctnnb1 via CreERT2 activation. (B-D) Fluorescent images showing the morphology of mesenteric collecting lymphatic vessels at postnatal day (P) 8. (E) Quantification of valves per millimeter at P8 and P14. (F-H) Fluorescent images showing the morphology of mesenteric collecting lymphatic vessels at P14. (I) Quantification of vessel length at P8 and P14. White arrowheads point to valves determined by high GFP (green) expression. 6 control, 5 Foxc2^{+/CreERT2} and 5 Foxc2^{+/creERT2}; Ctnnb1^{+/flox(exo3)} mesenteries are used for the analysis. All values are mean±SEM. One-way ANOVA was performed with Turkey's multiple comparisons for P8 and P14, respectively. ns=not significant, ****p<0.0001. Scale bars are 500 μM in B, C, D, F, G, and H.

FIG. 6. Immunostaining of valve genes, tight junction proteins, and SMA in P8 mesenteric vessels with postnatal deletion. Panels A-O show whole-mount immunostaining of P8 mesenteries with P1 TM injection from control, Foxc2^{+/CreERT2}, and Foxc2^{+/CreERT2}; Ctnnb1^{+/lox(ex3)} with Prox1 (green), Itga9 (purple), Foxc2 (red), Claudin 5 (gray), SMA (red), Itga9 (blue), and Prox1 (green). D, E, F, J, K, L are the higher magnification images of A, B, C, G, H, I. Scale bars are 100 μm. 4 controls and 4 knockouts were used in the analysis.

FIG. 7 shows the immunostaining antibodies used in the studies herein.

FIG. 8 shows the antibodies used for Western blots herein.

FIG. 9 Deletion of Foxo1 rescues valve loss in Cx43 knockout mice, which is a mouse model for Oculodentodigital dysplasia/lymphedema disease. Fluorescent images showing the morphology of mesenteric collecting lymphatic vessels at postnatal day (P) 14. White arrowheads point to valves determined by high GFP (green) expression.

### DETAILED DESCRIPTION

The disclosed methods may be utilized to treat diseases and disorders associated with loss of lymphatic valve tissue or lymphatic valve cell growth, which include, but are not limited to, congenital lymphedema.

In a first aspect, provided herein is a method for treating congenital lymphedema in a subject in need thereof, the method comprising administering to the subject a therapeutically effective amount of a FOXO1 inhibitor. Congenital lymphedema, also called Milroy disease, s a genetic disorder that causes fluid retention and tissue swelling in the lymphatic system. Congenital lymphedema may be present at birth, or develop up to age one. is a lymphatic disease commonly characterized by defects in lymphatic valve formation. Accordingly, as used herein, “a subject in need thereof” refers to a subject having been diagnosed with congenital lymphedema or expressing fluid retention and tissue swelling of the lymphatic system before the age of one year old. The subject may have lymphedema-distichiasis syndrome, a hereditary condition that causes a risk of swollen limbs due to problems in the lymphatic system, as well as a double row of eyelashes. Other symptoms such as cysts, light sensitivity, cardiac defects, cleft palate, astigmatism, and cornea scarring occur in some subjects. In embodiments, the subject has a primary lymphedema other than congenital lymphedema including, but not limited to, lymphedema praccox, lymphedema tarda, lymphedema-distichiasis syndrome, and Hennekam syndrome. The subject may be a human or a non-human mammal, such as a mouse or livestock animal. The subject may be no older than about 60 months old. The subject may at between just born (or 0 months old) and be between about and about 60 months old.

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof). “Treatment” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

As used herein, the term “administering” an agent, such as a FOXO1 inhibitor to a subject, is intended to refer to contacting, dispensing, delivering or applying the therapeutic agent to the subject by any suitable route for delivery of the therapeutic agent to the desired location in the animal, including delivery by either the parenteral or oral route, intramuscular injection, subcutaneous/intradermal injection, intravenous injection, intraperitoneal injection, buccal administration, transdermal delivery, topical administration, and administration by the respiratory tract route. In exemplary embodiments, the FOXO1 inhibitor is administered intraperitoneally.

The FOXO1 inhibitor may be any pharmacological or chemical inhibitor of FOXO1. In exemplary embodiments, the FOXO1 inhibitor is AS1842856, having the chemical structure

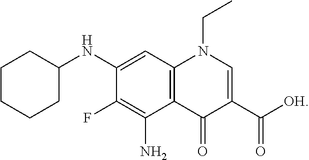 [SMILES: CCn1cc(C(=O)O)c(=O)c2c(N)c(F)c(NC3CCCCC3)cc21]

As used herein the terms “effective amount” and “therapeutically effective amount” refer to the amount or dose of the FOXO1 inhibitor that provides the desired effect. In some embodiments, the effective amount is the amount or dose of the FOXO1 inhibitor, upon single or multiple dose administration to the subject, which provides the desired effect in the subject under diagnosis or treatment. The disclosed methods may include administering an effective amount of the FOXO1 inhibitor (e.g., as present in a pharmaceutical composition) for treating congenital lymphedema or lymphedema-distichiasis syndrome. By way of example but not by way of limitation, in some embodiments an effective amount is sufficient to result in one or more of increased or restored lymphatic valve cell or tissue growth as compared to the subject before treatment, or compared to an appropriately matched, untreated control.

An effective amount can be readily determined by those of skill in the art, including an attending diagnostician, by the use of known techniques and by observing results obtained under analogous circumstances. In determining the effective amount or dose of FOXO1 inhibitor administered, a number of factors can be considered by the attending diagnostician, such as: the species of the subject; its size, age, and general health; the degree of involvement or the severity of the disease or disorder involved; the response of the individual subject; the particular FOXO1 inhibitor administered; the mode of administration; the bioavailability characteristics of the preparation administered; the dose regimen selected; the use of concomitant medication; and other relevant circumstances.

The AS1842856 may be administered at a dose between about 0.05 mg/kg and about 10 mg/kg, or any dose or range in between. In preferred embodiments, when the subject is a non-human mammal, the AS1842856 is administered at a dose between about 4 mg/kg to about 10 mg/kg. In exemplary embodiments, the AS1842856 is administered at a dose of about 8 mg/kg. In preferred embodiments, when the subject is a human, the AS1842856 is administered at a dose between about 0.05 mg/kg and about 0.5 mg/kg.

The FOXO1 inhibitor may be administered more than once and or in combination with other methods of standard of care. In exemplary embodiments the FOXO1 inhibitor, AS1842856, is administered at about every other day.

The methods provided herein may further comprise administering to a subject in need an additional therapy to alleviate any of the above symptoms.

In a second aspect, provided herein is a method for increasing or restoring lymphatic valve growth in a subject in need thereof, comprising administering a FOXO1 inhibitor. As used herein, “a subject in need thereof” refers to a subject having compromised lymphatic valve growth. The compromised lymphatic valve growth may be due to a disease or disorder, such as congenital lymphedema or lymphedema-distichiasis syndrome. In embodiments, the subject has a primary lymphedema other than congenital lymphedema including, but not limited to, lymphedema praecox, lymphedema tarda, lymphedema-distichiasis syndrome, and Hennekam syndrome. The subject may be a human or a non-human mammal. The subject may be between about 0 and about 60 months old.

The FOXO1 inhibitor may be administered by any appropriate route, e.g. the parenteral or oral route, intramuscular injection, subcutaneous/intradermal injection, intravenous injection, intraperitoneal injection, buccal administration, transdermal delivery, topical administration, and administration by the respiratory tract route. In exemplary embodiments, the FOXO1 inhibitor is administered intraperitoneally.

The FOXO1 inhibitor may be any pharmacological or chemical inhibitor of FOXO1. In exemplary embodiments, the FOXO1 inhibitor is AS1842856. The AS1842856 may be administered at a dose between about 0.05 mg/kg and about 10 mg/kg, or any dose or range in between. In preferred embodiments, when the subject is a non-human mammal, the AS1842856 is administered at a dose between about 4 mg/kg to about 10 mg/kg. In exemplary embodiments, the AS1842856 is administered at a dose of about 8 mg/kg. In preferred embodiments, when the subject is a human, the AS1842856 is administered at a dose between about 0.05 mg/kg and about 0.5 mg/kg.

The FOXO1 inhibitor may be administered more than once and or in combination with other methods of standard of care. In exemplary embodiments the FOXO1 inhibitor, AS1842856, is administered at about every other day. The methods provided herein may further comprise administering to a subject in need an additional therapy to alleviate any of the above symptoms.

In a third aspect, provided herein is a method for increasing nuclear β-catenin activity in a cell, the method comprising contacting the cell with a FOXO1 inhibitor. As demonstrated herein, FOXO1 inhibitor AS1842856 increases the expression of valve genes in hdLECs by inhibiting FOXO1 and increasing β-catenin activity in the nucleus.

“Contacting” a cell refers to adding an agent, e.g. a FOXO1 inhibitor to a culture medium or buffer containing the cell.

The cell may be a lymphatic endothelial cell. The cell may be a primary cell or an immortalized cell line. The cell may be a human cell or a non-human mammalian cell. In exemplary embodiments the cell is a human dermal lymphatic endothelial cell.

The FOXO1 inhibitor may be AS1842856. The cell may be contacted with the FOXO1 inhibitor, e.g. AS1842856, for about 48 hours. The FOXO1 inhibitor, e.g. AS1842856, concentration may be between about 0.1 μM and about 1 μM. In exemplary embodiments, the AS1842856 concentration is about 0.5 μM.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The term “subject” may be used interchangeably with the terms “individual” and “patient” and includes human and non-human mammalian subjects.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following Examples are illustrative and should not be interpreted to limit the scope of the claimed subject matter.

The inventors investigated the effect of the pharmacological inhibition of Foxo1 using a specific inhibitor AS1842856 in cultured human dermal lymphatic endothelial cells (hdLECs) and in mice. The data shows that hdLECs treated with AS1842856 had increased expression levels of valve-forming genes, including FOXC2, GATA2, KLF4, ITGA9, and GJA4 on both mRNA and protein levels. Intriguingly, more β-catenin was accumulated in the nucleus of hdLECs treated with AS1842856, compared to the vehicle control, which stimulates valve gene expression. Furthermore, genetic overactivation of β-catenin in the nucleus rescued the valve loss in Foxc2 heterozygous mice to the wildtype level. Meanwhile, treatment of AS1842856 in Foxc2 heterozygous mice significantly increased the number of valves. Therefore, besides genetic deletion of Foxo1, valve defects in a primary lymphedema mouse model can also be rescued by using a drug to pharmacologically inhibit Foxo1.

### Results

### AS1842856 Treatment Results in the Upregulation of Valve Genes in a Time and Concentration Dependent Manner

The transcription factor Foxo1 is expressed in many cell types in the body, including lymphatic endothelial cells (LECs). The inventors have previously reported that Foxo1 acts as a repressor for lymphatic valve formation. Deletion of Foxo1 in LECs induces the growth of new valves in vivo. More importantly, ablation of Foxo1 in a mouse model for human disease lymphedema-distichiasis, the Foxc2 heterozygous mice, completely rescues both the loss of valves and the leakiness of valves in this disease model. Mechanistically, knockdown of FOXO1 using shRNA in human dermal lymphatic endothelial cells (hdLECs) markedly upregulates the expression of valve-forming genes, including FOXC2, GATA2, and KLF4. Furthermore, Foxo1 mediates the repression activity by directly binding to the promoter region of FOXC2 gene and negatively regulates FOXC2 expression (Scallan et al., 2021). These data strongly indicate that FOXO1 is a potential target for treating lymphedema-distichiasis.

AS1842856 (AS) is a cell permeable inhibitor of Foxo1 and has minimal effect on inhibiting the activity of FOXO3 and FOXO4 (Nagashima et al., 2010). Several research groups have reported that the function of Foxo1 can be pharmacologically inhibited by AS1842856 in different cell types (Zou et al., 2014, Jeon et al., 2018, Wang et al., 2020, Jeon et al., 2021). Therefore, the inventors investigated the effect of AS1842856 treatment on Foxo1 activity and the expression of valve genes in hdLECs.

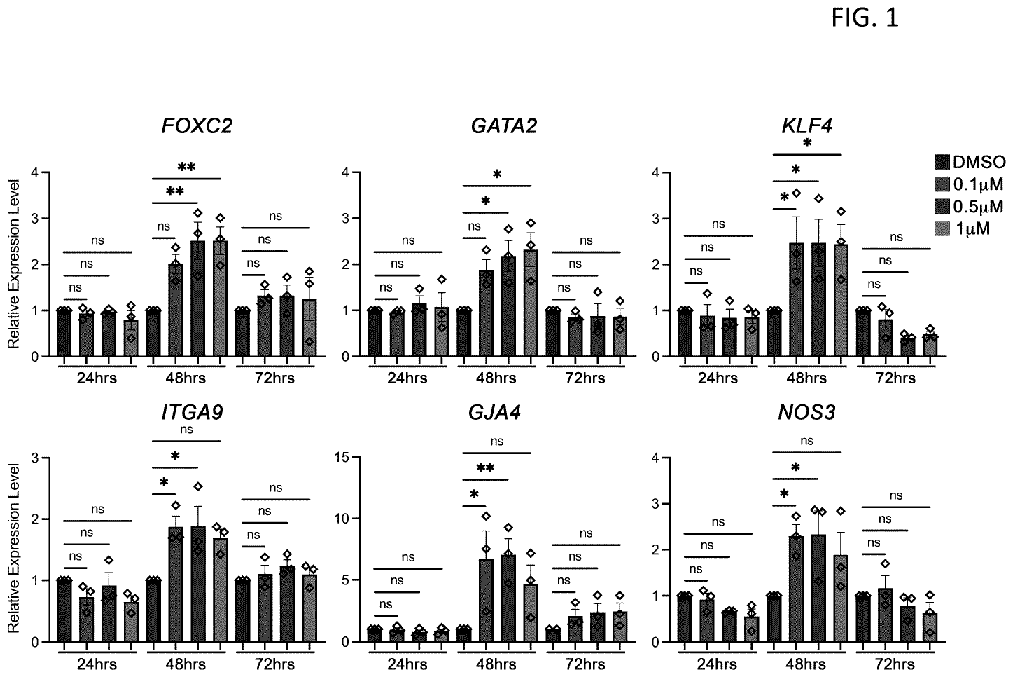

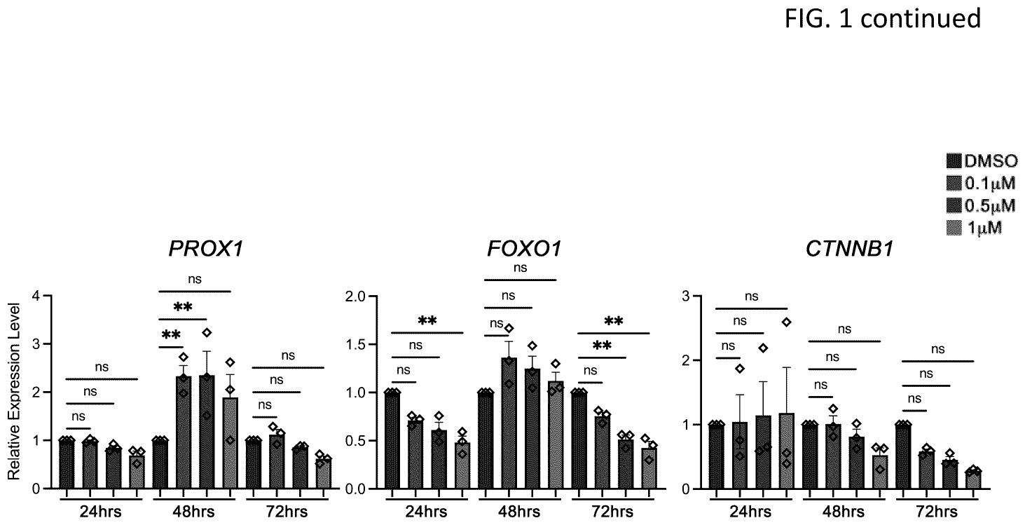

To investigate this, hdLECs were treated with three distinct concentrations of AS1842856-0.1 μM, 0.5 μM, and 1 μM for 24 hours (hrs), 48 hrs, and 72 hrs. DMSO was used as the vehicle control. The medium was changed every day with freshly made drug. Thereafter, the expression levels of valve genes and flow responsive genes, including FOXC2, GATA2, PROX1, KLF4, ITGA9, GJA4, NOS3, and CTNNB1, were determined using quantitative RT-qPCR. Specifically, transcription factors FOXC2, GATA2, and CTNNB1 (β-catenin) have been shown to be essential for lymphatic valve formation. Deletion of any of these three genes leads to the absence of lymphatic valve formation during embryonic development (Petrova et al., 2004, Kazenwadel et al., 2015, Cha et al., 2016). Quantitative RT-PCR results showed that the expression levels of FOXC2 and GATA2 were significantly upregulated with 48 hrs AS treatment, but not with 24 hrs or 72 hrs treatment (FIG. 1). However, the expression of CTNNB1 was not altered. Meanwhile, the expression levels of the key flow responsive gene KLF4 and the master LEC gene PROX1 were significantly increased under the same condition.

Additionally, it is reported that both ITGA9 (Integrin α9) and GJA4 (Connexin37) are highly expressed in the valve leaflets. Loss-of-function of either gene results in defective valves (Bazigou et al., 2009, Kanady et al., 2011). Here, AS treatment for 48 hrs induced significantly higher expression levels of ITGA9 and GJA4 (FIG. 1). NOS3 (eNOS) is a downstream gene in the mechanotransduction pathway in blood endothelial cells (BEC), which is expressed in lymphatic valve cells and is regulated by FOXC2 in LECs (Sabine et al., 2012). Here, NOS3 expression was significantly increased with 48 hrs AS treatment as well. Furthermore, as expected, the expression level of FOXO1 was not altered by AS treatment because AS1842856 does not affect either the transcription or the translation of FOXO1. Instead, AS1842856 binds to FOXO1 protein to prevent the binding of FOXO1 to the promoter regions and reduces the transcription activity of FOXO1 in the nucleus (Nagashima et al., 2010). In summary, the expression pattern of valve genes with respect to time showed a significant increase at 48 hrs treatment of AS1842856, but not at 24 hrs or 72 hrs (FIG. 1).

In addition to testing the length of the treatment, three concentrations of AS1842856-0.1 μM, 0.5 μM, and 1 μM were used. Since 48 hrs treatment was the only time point to show the expression changes in valve genes, the inventors focused on 48 hrs AS treatment in further experiments. Although the expression levels of FOXC2 and GATA2 displayed a trend of increase under 0.1 μM treatment, both the 0.5 μM and 1 μM AS treatment induced a significant increase in the expression of FOXC2 and GATA2. Meanwhile, KLF4 expression was increased by the treatment of all three concentrations of AS. Furthermore, the expression levels of ITGA9, GJA4, NOS3, and PROX1 were upregulated only under 0.1 μM and 0.5 μM, but not under 1 μM. None of the concentrations induced significant upregulation of either FOXO1 or CTNNB1 expression (FIG. 1). Overall, the quantitative RT-PCR results showed that the FOXO1 inhibitor AS1842856 induced the expression of valve genes in a time and concentration dependent manner. Treatment of 0.5 μM of AS1842856 for 48 hrs provided the best results for the increased expression of FOXC2, GATA2, KLF4, ITGA9, GJA4, NOS3, and PROX1.

AS1842856 Treatment Increases the Nuclear Activity of β-Catenin in hdLECs

Treatment of 0.5 μM of AS1842856 for 48 hrs significantly increased the expression levels of valve genes on the mRNA level. Next, the inventors performed western blot on the cells treated with 0.5 μM for 48 hours to determine whether the expression of these genes on the protein level was consistent with the PCR results. Three independent experiments of cultured hdLECs were performed, similarly to the quantitative RT-qPCR experiments. Protein levels of FOXC2, GATA2, KLF4, ITGα9, GJA4, activated β-catenin, total β-catenin, PROX1, and FOXO1 were quantified from western blot.

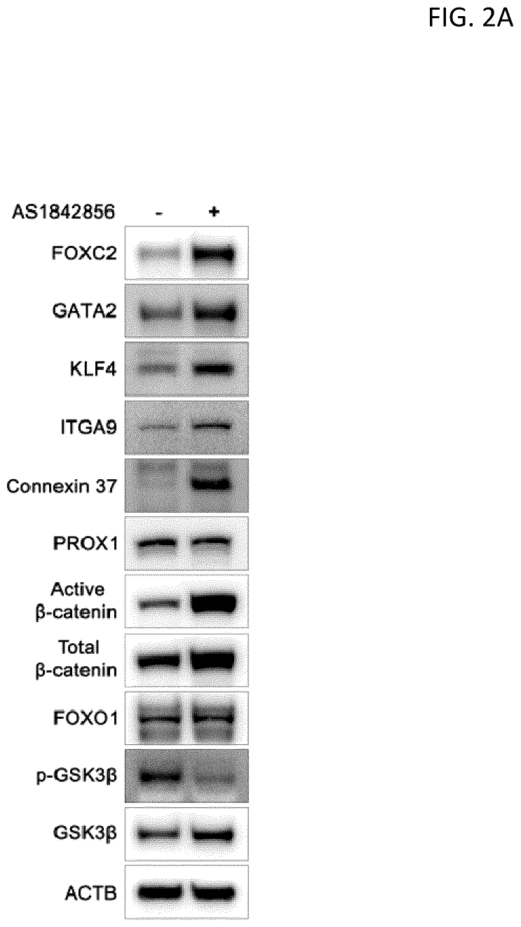

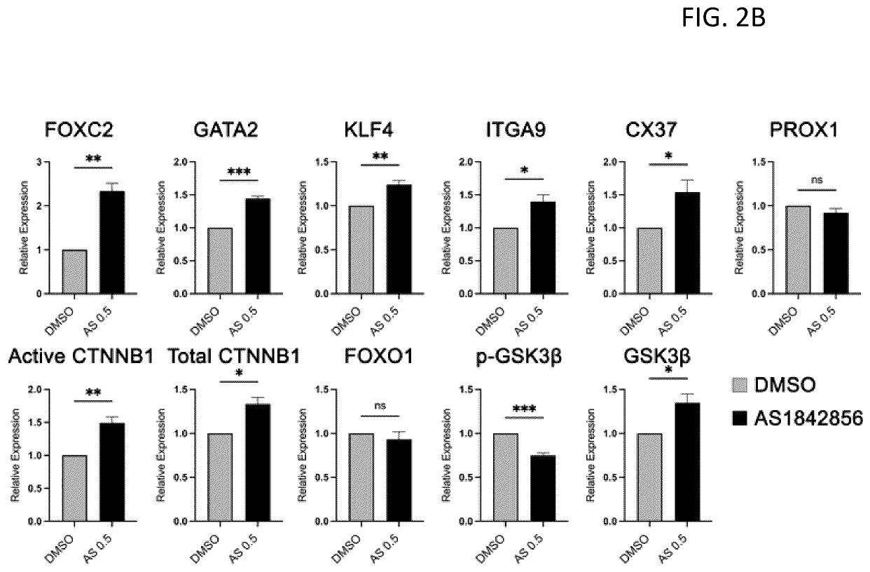

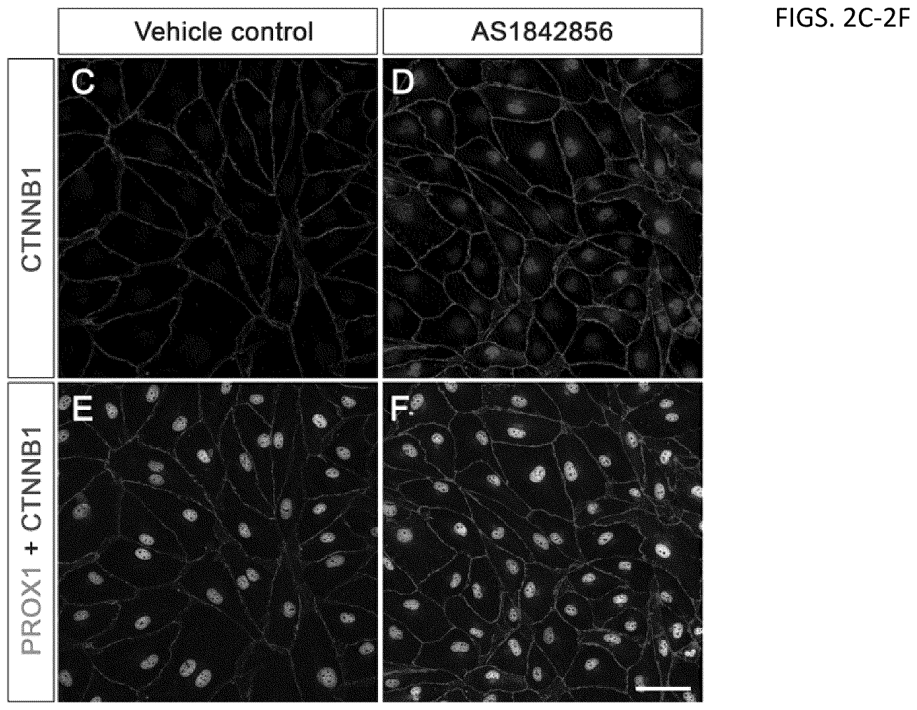

As expected, the protein expression levels of FOXC2, GATA2, KLF4, ITGA9, and GJA4 were significantly upregulated upon AS treatment compared to the vehicle control while FOXO1 protein expression did not change in response to AS treatment (FIG. 2A), which was consistent with the results from the mRNA expression (FIG. 2B). However, the protein level of PROX1 did not alter although the mRNA level of PROX1 was increased. Notably, the amount of active β-catenin and total β-catenin protein increased significantly in AS treated cells (FIG. 2B). Since the active β-catenin antibody only detects the non-phosphorylated form of b-catenin, which is the pool of β-catenin that is not phosphorylated by glycogen synthase kinase 3beta (GSK3β, one of the key components of the destruction complex) to be degraded by the destruction complex, the western blot results suggest that AS1842856 treatment could inhibit the degradation of β-catenin by reducing β-catenin phosphorylation to increase the amount of non-phosphorylated β-catenin. Intriguingly, the protein amount of total β-catenin also increased in the AS treatment despite the unchanged mRNA level of β-catenin in the same condition. This further confirmed that AS1842856 regulated the amount of β-catenin on a post-transcriptional level.

To investigate whether the activity of the β-catenin destruction complex was decreased in response to AS1842856 treatment, the levels of GSK3β and its phosphorylated form (i.e., the inactive form) p-GSK3β were analyzed in the Western blot. Unexpectedly, the amount of total GSK3β was significantly increased and the amount of p-GSK3β was significantly decreased (FIG. 2B). These results indicate that AS1842856 treatment increases the nuclear activity of β-catenin through different mechanisms other than inhibiting the destruction complex through GSK3B.

In cells, phosphorylated β-catenin is degraded in a destruction complex in the cytoplasm, but unphosphorylated β-catenin upon activation is translocated to the nucleus from the cytoplasm (Harada et al., 1999). It has been reported that β-catenin in the nucleus of LECs promotes the expression of valve-forming genes FOXC2 and PROX1 by binding to the promoter areas of these two genes (Cha et al., 2016). Since AS1842856 treatment increased the amount of activated (non-phosphorylated) β-catenin in western blot (FIG. 2A), the inventors performed immunostaining of the LEC transcription factor PROX1 and β-catenin in hdLECs treated with 0.5 μM AS for 48 hours to visualize the localization of β-catenin. Under the control condition (DMSO), β-catenin was almost exclusively localized on the cell membrane in the vehicle treated hdLECs while PROX1 is only in the nucleus (FIGS. 2C and E). Strikingly, a remarkable amount of β-catenin was in the nucleus of AS treated cells in addition to the cell membrane (FIG. 2D). Merged confocal images showed the colocalization of PROX1 and β-catenin in the nucleus of AS treated cells (FIG. 2F). These data indicated that 1) the nuclear β-catenin seen in immunostaining represented the non-phosphorylated β catenin shown in western blot; 2) AS1842856 treatment increased the nuclear activity of β-catenin in LECs, which would upregulate the expression of valve genes, such as FOXC2. Collectively, the inventors have shown that AS1842856 increased the expression of valve genes at the mRNA and protein levels in a concentration and time-dependent manner in cultured hdLECs by inhibiting FOXO1 activity and by increasing β-catenin activity in the nucleus.

### AS1842856 Treatment Increases Valve Number in Foxc2 Heterozygous Mice

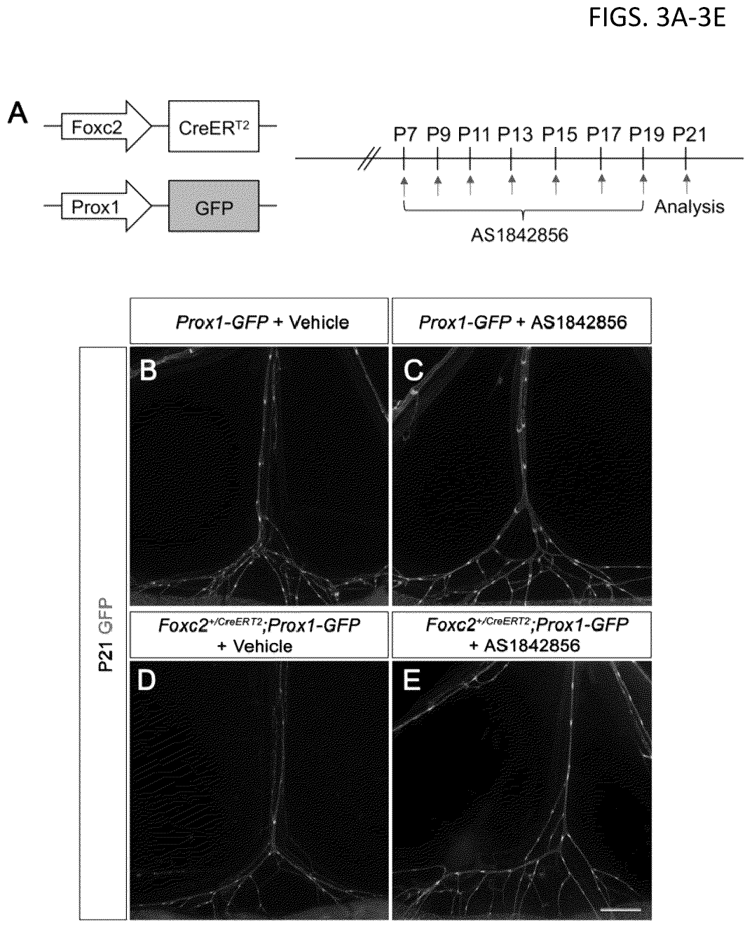

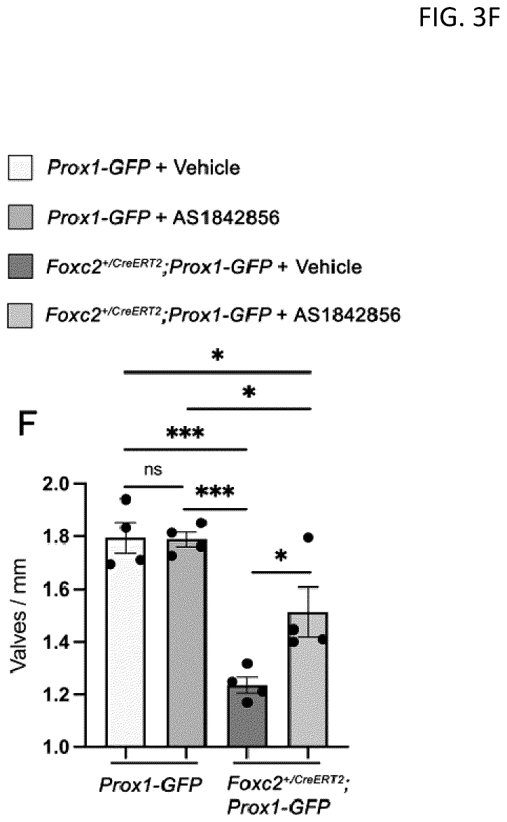

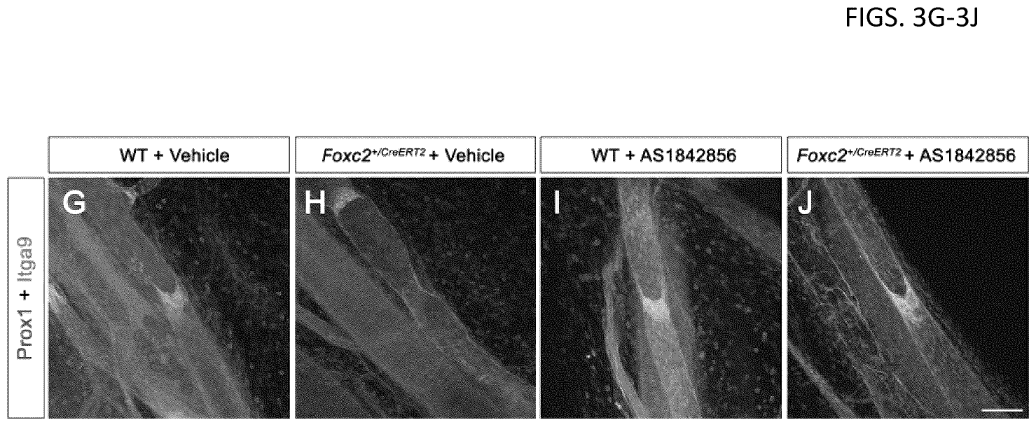

Foxc2 is a chief regulator of lymphatic valve development modulating the expression of many valve genes (Sabine et al., 2015). While Foxc2 null animals are embryonic lethal, the Foxc2 heterozygous mice are a model of the human disease lymphedema-distichiasis with severe lymphatic valve loss (Kriederman et al., 2003, Petrova et al., 2004). To test whether AS1842856 treatment increases valve growth in vivo, the inventors intraperitoneally (i.p.) injected AS1842856 into Foxc2^{+/CreERT2} pups. Instead of using wildtype pups as control, Prox1-GFP mice were used as control as well as for visualizing the lymphatic vasculature (Choi et al., 2011). The Foxc2^{+/CreERT2} pups were also bred to carry the Prox1-GFP allele. Since the open reading frame in one allele of the Foxc2 gene is replaced with the cDNA for CreERT2, the Foxc2^{+/CreERT2} mice is heterozygous for Foxc2 gene (Geng et al., 2016). Four groups of pups were used: 1) Prox1-GFP pups with DMSO (vehicle control) injection; 2) Prox1-GFP pups with AS1842856 injection; 3) Foxc2^{+/CreERT2}; Prox1-GFP pups with DMSO injection; and 4) Foxc2^{+/CreERT2}; Prox1-GFP pups with AS1842856 injection. DMSO and AS1842856 injections were performed every other day from P7-P21 (FIG. 3A). To compare the number of valves from these 4 groups, the inventors counted total number of valves from 6 branches (from the lymph nodes to the gut wall) in the mesentery and measured the total length of these branches as previously published (Scallan et al., 2021). The average valve number per mm (valves/mm) was used for comparison. Quantitative results showed that 1) valves/mm was significantly lower in Foxc2^{+/CreERT2} pups with DMSO injection than Prox1-GFP pups with DMSO injection; 2) valves/mm was significantly higher in Foxc2^{+/CreERT2} pups with AS1842856 injection than in Foxc2^{+/CreERT2} pups with DMSO injection; 3) valves/mm was still significantly lower in Foxc2^{+/CreERT2} pups with AS1842856 injection than Prox1-GFP pups with DMSO injection; and 4) there was no significant difference in valves/mm between Prox1-GFP pups with DMSO injection and Prox1-GFP pups with AS1842856 injection (FIGS. 3B-F). Whole-mount immunostaining of Prox1 and Itga9 was performed to visualize the morphology of valves with and without AS1842856 treatment in wildtype and Foxc2^{+/CreERT2} pups (FIGS. 3G-J). The data showed that 1% of the valve areas with high Prox1 expression in the Foxc2^{+/CreERT2} pups did not express Itga9 (FIG. 3H), which is consistent with the inventors' previous publication (Scallan et al., 2021) and suggests that these valves are not mature. However, all the valves from AS1842856 treated Foxc2^{+/CreERT2} pups expressed Itga9.

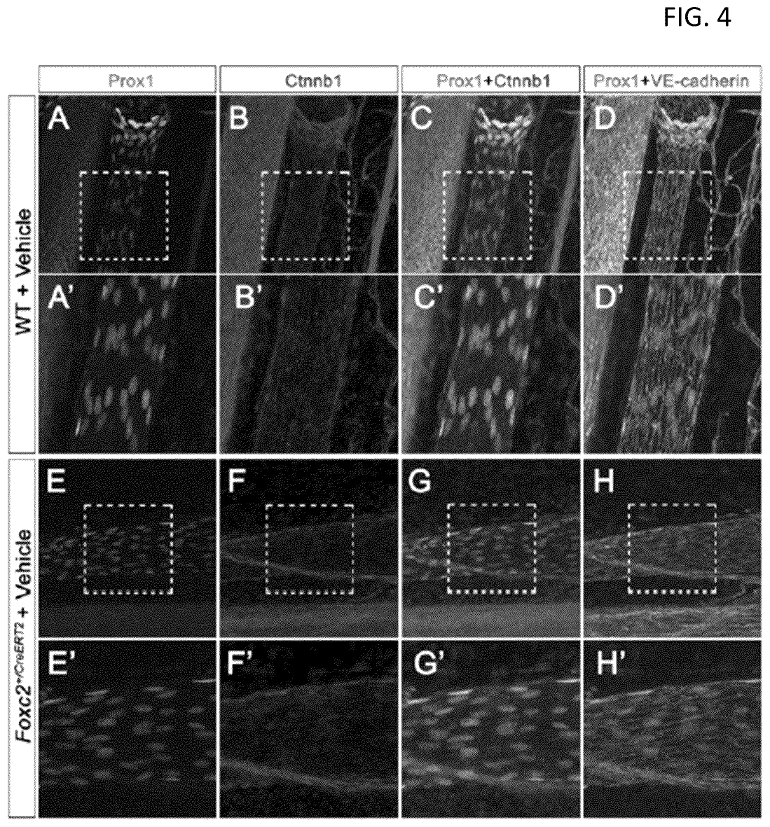

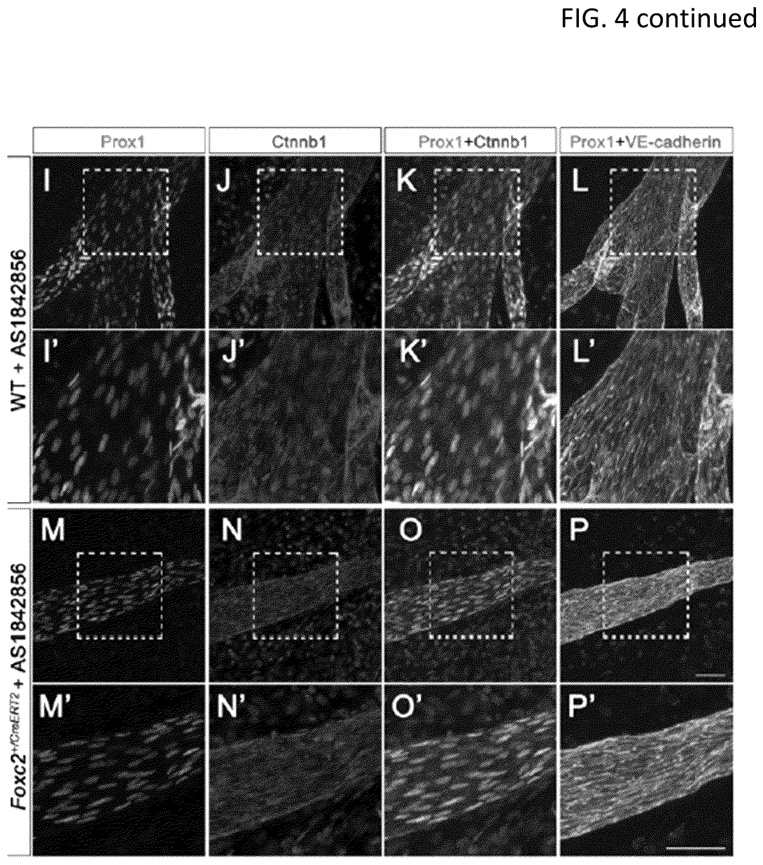

To further demonstrate the effect of AS1842856 injection on the nuclear localization of β-catenin in vivo, the inventors whole-mount immunostained Prox1 in green, β-catenin in purple, and VE cadherin in gray on the mesenteries after two injections of DMSO or AS1842856 (FIG. 4, panels A-P′). β-catenin was mostly localized on the cell membrane in the DMSO injected group, indicated by the strong nuclear green Prox1 staining in the Prox1 and β-catenin merged images (FIG. 4C, panels C′, G, and G′). In contrast, due to the increased nuclear purple staining from β-catenin in the AS1842856 injected group, the nuclei became gray colored in the Prox1 (green) and β-catenin (purple) merged images (FIG. 4, K, panels K′, O, and O′). In conclusion, AS1842856 injection significantly increased lymphatic valves in Foxc2^{+/CreERT2} pups, but did not rescue the defect to the wildtype level.

### Increased Nuclear Activity of β-Catenin In Vivo Restores the Valve Loss in Foxc2 Heterozygous Mice

The inventors' results show that AS1842856 treatment increased the nuclear activity of β-catenin in both cultured hdLECs and in vivo, and AS1842856 injection in vivo led to the growth of new valves in Foxc2 heterozygous pups. Furthermore, it is reported that β-catenin directly binds to the FOXC2 promoter region to activate the expression of FOXC2 (Cha et al., 2016). It indicates that more nuclear β-catenin induced by Foxo1 inhibition could partially contribute to the growth of new valves. Therefore, the inventors examined whether increased activity of b-catenin in the nucleus would restore the valve loss in Foxc2 heterozygous mice.

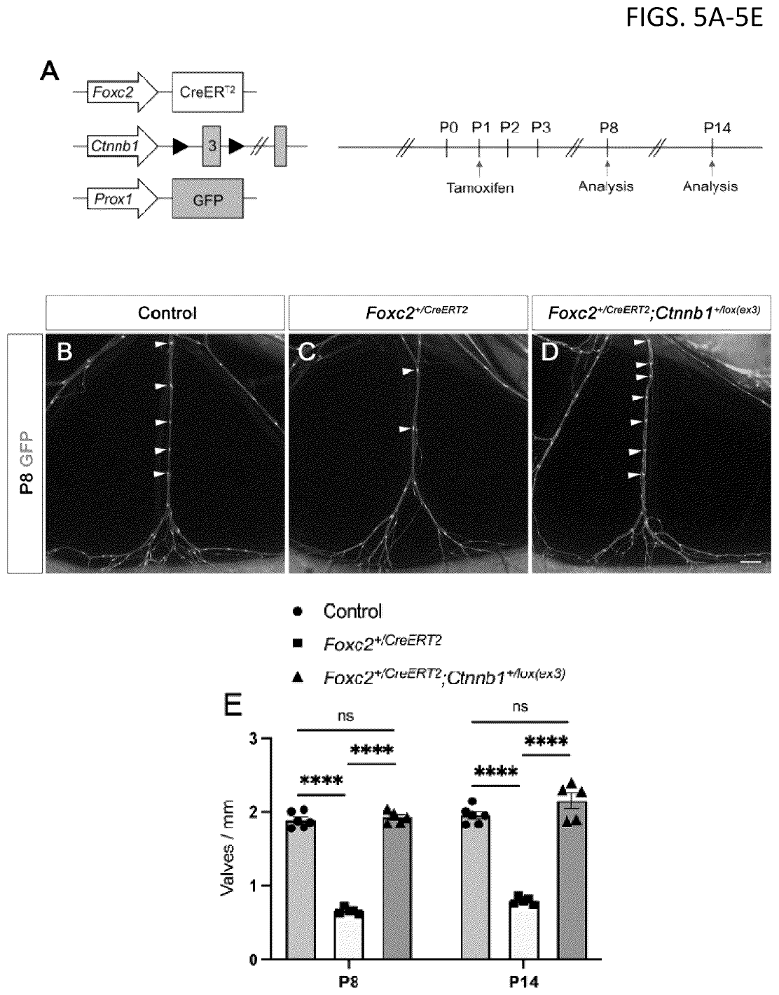

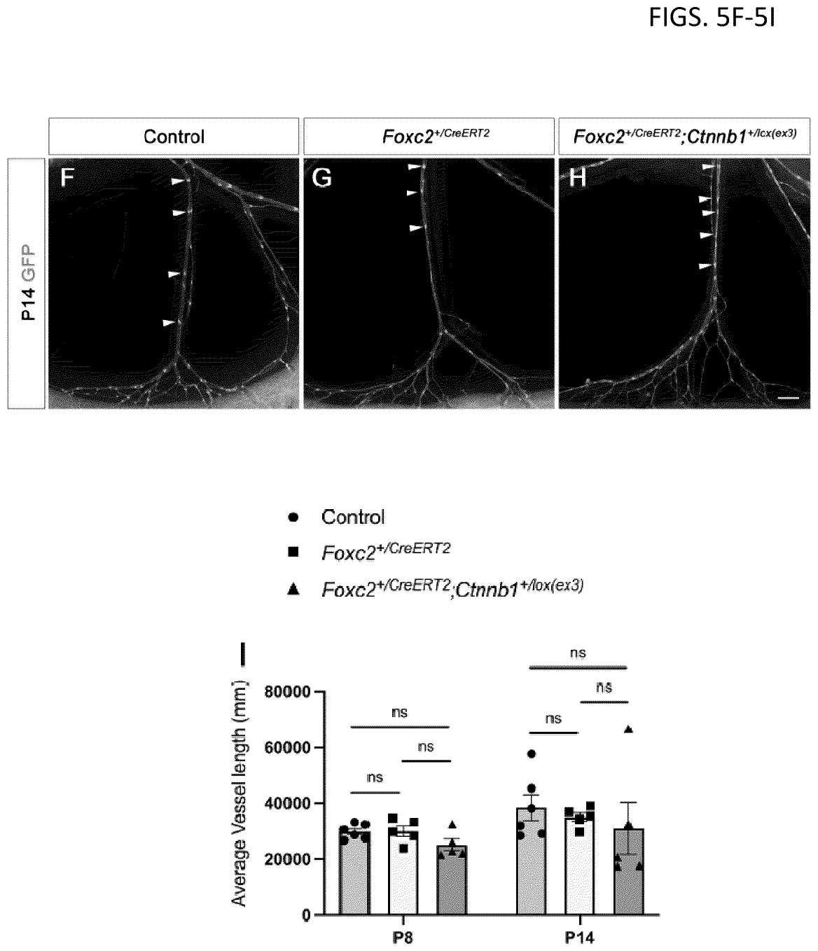

To increase the nuclear activity of β-catenin in vivo, the inventors used Ctnnb1^{lox(ex3)} mice, in which the serine and threonine encoded by exon 3 of the β-catenin gene (Ctnnb1) and phosphorylated by GSK3β is deleted to prevent the degradation of β-catenin in the destruction complex (Harada et al., 1999). The inventors bred the Foxc2^{+/CreERT2} mice with the Ctnnb1^{lox(ex3)} mice and the LEC reporter line, Prox1-GFP, for visualizing the lymphatic vasculature (Choi et al., 2011) to obtain the Foxc2^{+/CreERT2}; Ctnnb1^{lox(ex3)}; Prox1-GFP mice. As mentioned above, the open reading frame in one allele of the Foxc2 gene is replaced with the cDNA for CreER^{T2} in the Foxc2^{+/CreERT2} mice making it heterozygous for Foxc2 and carrying tamoxifen inducible Cre activity to recombine loxp sites. The inventors injected tamoxifen (TM) at postnatal day (P) 1 and analyzed the mesenteric lymphatic vasculature at P8 and P14 in the Foxc2^{+/CreERT2}; Prox1-GFP; the Foxc2^{+/CreERT2}; Ctnnb1^{lox(ex3)}; Prox1-GFP and the control Ctnnb1^{lox(ex3)}; Prox1-GFP mice (FIG. 5A). The valves/mm and the average vessel length were used for comparison. The morphology of the lymphatic vasculature shown by the GFP images demonstrated that the distance between two adjacent valves was longer in the Foxc2^{+/CreERT2}; Prox1-GFP mesentery compared to either the control or the Foxc2^{+/CreERT2}; Ctnnb1^{lox(ex3)}; Prox1-GFP mice (FIGS. 5B-D). To support this finding, quantitative data confirmed that valves/mm was significantly reduced in Foxc2^{+/CreERT2}; Prox1-GFP mice at P8 compared to the control mesentery (FIG. 5E). The vessel length was not significantly different between the three genotypes (FIG. 5I). Remarkably, the valve loss in the Foxc2^{+/CreERT2}; Prox1-GFP mice was completely rescued by the increased nuclear activity of β-catenin in the Foxc2^{+/CreERT2}; Ctnnb1^{lox(ex3)}; Prox1-GFP mice (FIG. 5E). Moreover, this rescue phenotype was consistent in the P14 mesentery. The significantly reduced valves/mm in the Foxc2^{+/CreERT2}; Prox1-GFP mesentery was restored to control levels in the Foxc2^{+/CreERT2}; Ctnnb1^{lox(ex3)}; Prox1-GFP mice at P14 (FIGS. 5E-I). In summary, the data revealed that enhancing the activity of β-catenin in the nucleus of LECs can induce the growth of new valves, which can rescue the valve loss caused by impaired development of valves in the Foxc2 heterozygous mice, a congenital lymphedema mouse model.

Valves in Foxc2^{+/CreERT2}; Ctnnb1^{lox(ex3)} Mice Express Valve Genes and Exhibit Normal Junctions

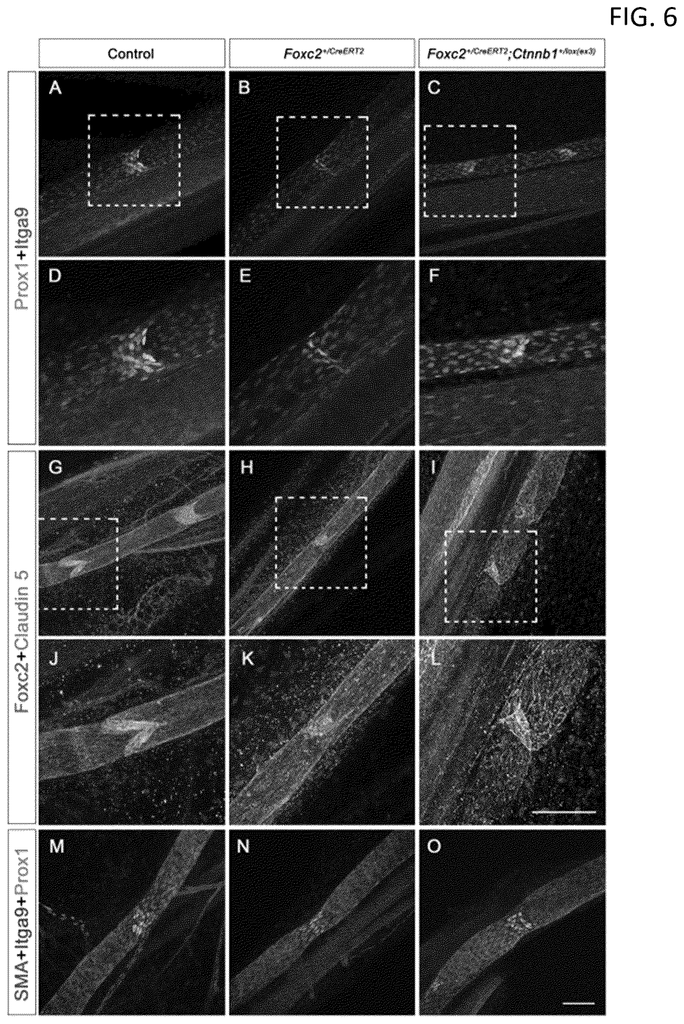

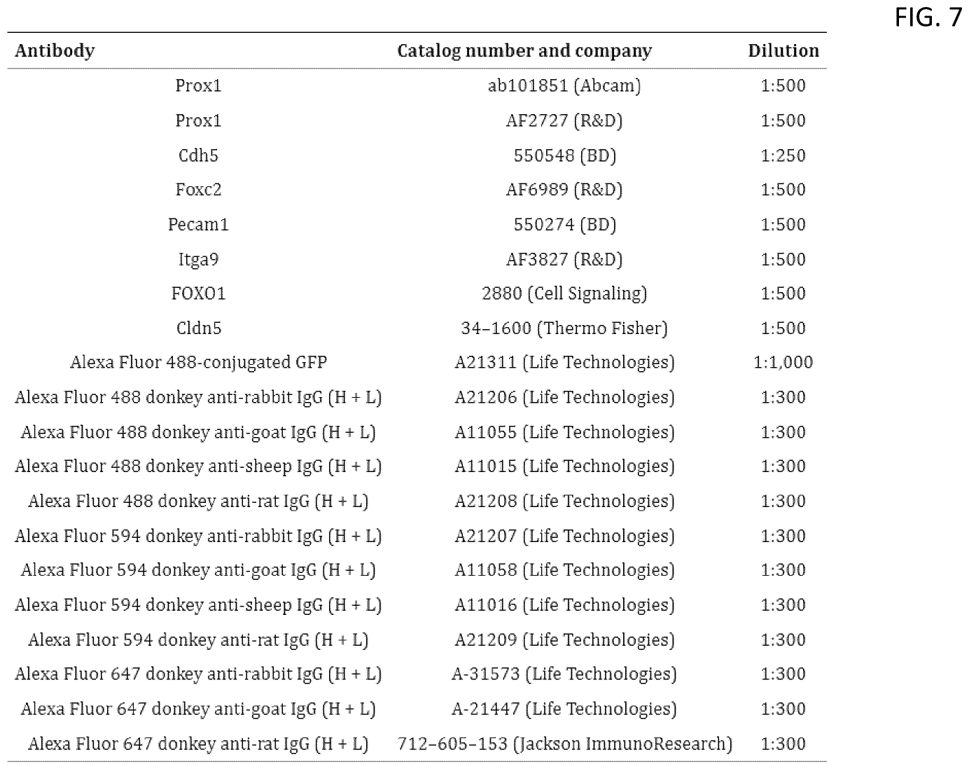

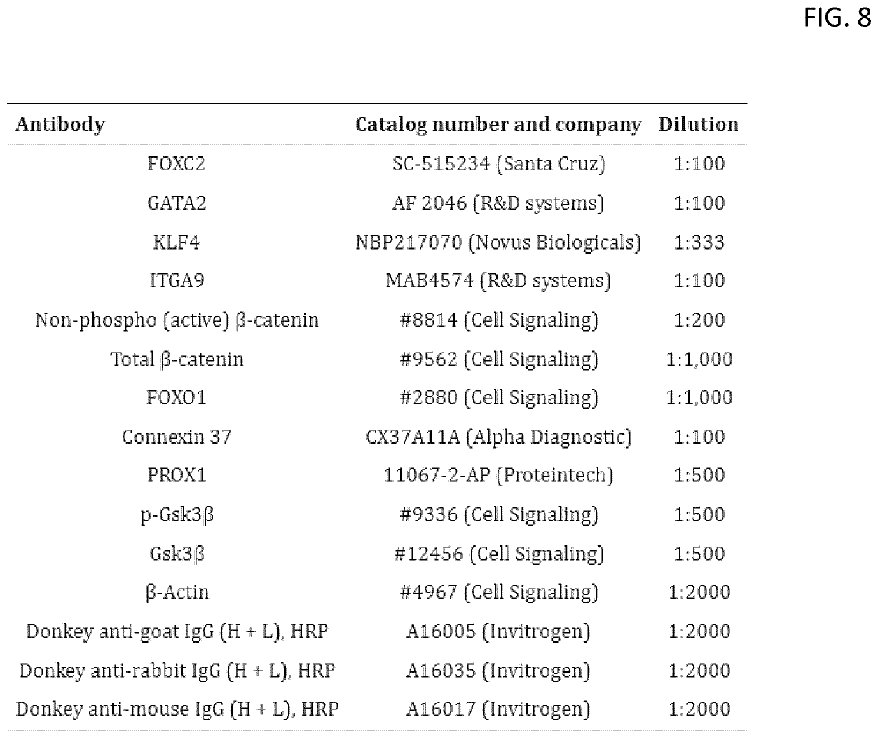

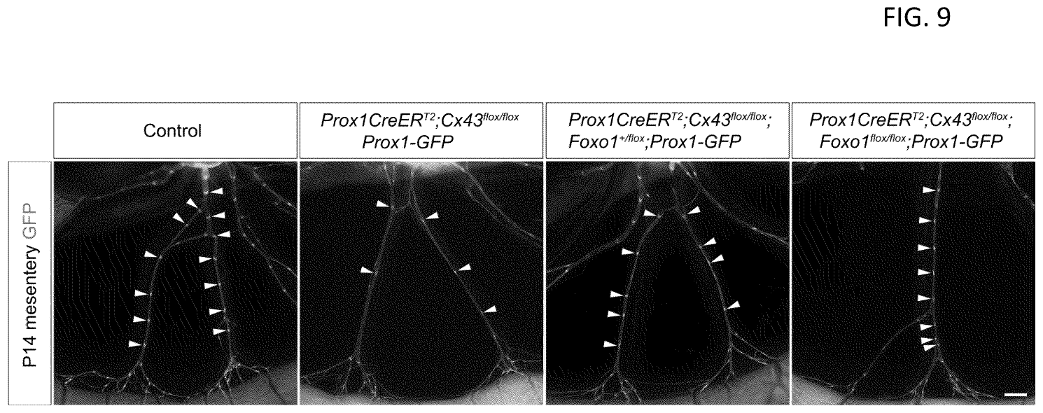

The inventors have shown that increased β-catenin in the nucleus induces lymphatic valves to grow. In order to determine whether those valves in the Foxc2^{+/CreERT2}; Ctnnb1^{lox(ex3)} mesentery expressed the same valve genes as the controls, the same TM protocol was performed as in FIG. 4A, and P8 mesentery was used for immunostaining. Since both Prox1 and Foxc2 are upregulated in the valve cells during valve formation while Itga9 is specifically expressed the valve leaflets, whole-mount immunostaining of valve genes Prox1, Foxc2, and Itga9 was performed in P8 mesenteries from the control, the Foxc2^{+/CreERT2}, and the Foxc2^{+/CreERT2}; Ctnnb1^{lox(ex3)} mice. Multiple areas in the mesenteric lymphatic vessels were imaged in the Foxc2^{+/CreERT2}; Ctnnb1^{lox(ex3)} mice and the expression pattern of those valve genes to the control and Foxc2^{+/CreERT2} were compared. While all the valves in both the control and the Foxc2^{+/CreERT2}; Ctnnb1^{lox(ex3)} mesentery expressed high levels of Prox1, Foxc2, and Itga9, 1% of Prox1^{high} valve areas in Foxc2^{+/CreERT2} mice lacked the expression of Itga9, indicating that these valve areas were missing the valve leaflets, which is consistent with the inventors' previous findings (Scallan et al., 2021) (FIG. 6, panels A-F). Therefore, the data confirmed that the loss of valve leaflets indicated by Itga9 staining in Foxc2^{+/CreERT2} was restored by increasing nuclear activity of β-catenin.

In addition to valve genes, tight junction protein Claudin 5 was immunostained. The collecting lymphatic vessels from all three genotypes had continuous junctions displayed by the expression of Claudin 5 (Cldn5) (FIG. 6, panels G-L). Therefore, increased activity of β-catenin in the nucleus did not disrupt the formation of junctions. No lymph leakage was observed in these animals.

Mature collecting lymphatic vessels have abundant smooth muscle cell (SMC) coverage (Oliver, 2004). Loss of SMC recruitment indicates defective formation and maturation of the collecting lymphatic vessels (Breslin et al., 2018). To test whether more β-catenin in the nucleus affects SMC recruitment, the inventors immunostained the mesentery with smooth muscle alpha-actin (SMA) in the control, the Foxc2^{+/CreERT2}, and the Foxc2^{+/CreERT2}; Ctnnb1^{lox(ex3)} mice at P14. SMA staining showed that SMC were recruited to the collecting lymphatic vessels in all three genotypes and the increased nuclear activity of β-catenin in the nucleus did not affect SMC recruitment (FIG. 6, panels M-O). Thus, the increased nuclear activity of β-catenin in LEC specifically induced the formation of lymphatic valves in the Foxc2 heterozygous mice and rescued the valve loss in this mouse model of lymphedema-distichiasis without altering the expression of junction proteins and SMC coverage.

### Discussion

Previously, it was shown that deletion of Foxo1 in the lymphatic endothelium induces new lymphatic valves to grow, which rescues the lymphatic valve defects in the Foxc2 heterozygous mice, a mouse model of lymphedema-distichiasis by increasing the number of valves and by enhancing the function of valves. It was the first study to show that valve defects in congenital lymphedema can be restored after the impaired lymphatic vasculature has formed, which drove the inventors to investigate further in the function of Foxo1 inhibition in treating lymphedema. Here, the inventors tested the effect of a Foxo1 specific inhibitor AS1842856 in hdLECs and found that AS1842856 treatment upregulated the expression of valve and flow responsive genes, including FOXC2, GATA2, KLF4, ITGA9, GJA4, and NOS3 on both the mRNA and protein levels. Moreover, i.p injection of AS1842856 increased the valve number in Foxc2 heterozygous mice in vivo. Intriguingly, western blot showed that AS1842856 treatment increased the level of non-phosphorylated β-catenin, the active form of β-catenin, which was visualized by immunostaining in the nucleus of both cultured hdLECs and mouse mesenteric LEC in vivo. These data indicated that AS1842856 treatment enhanced the nuclear activity of β-catenin in LECs. To further investigate the function of nuclear β-catenin in vivo, the inventors genetically increased the activity of β-catenin in the nucleus of LEC in Foxc2 heterozygous mice, a mouse model of lymphedema distichiasis. Strikingly, enhanced nuclear β-catenin restored the valve loss in Foxc2 heterozygous mice to the wildtype level. Therefore, in addition to deletion of Foxo1, activation of β-catenin in the nucleus of LECs induces new lymphatic valves to grow.

### AS1842856 Treatment Increases the Expression of Lymphatic Valve Genes

Lymphatic valve formation requires activated signaling from oscillatory shear stress (OSS) from lymph (Sweet et al., 2015). It has been shown that OSS treatment in LECs upregulates the expression of key valve-forming transcription factors Foxc2 and Gata2. Although the expression level of Prox1 in cultured LECs does not change in response to flow treatment, Foxc2, Prox1, and Gata2 have been described as key transcription factors in vivo to begin valve development and they cooperatively regulate each other in a feed forward mechanism (Sabine et al., 2015, Kazenwadel et al., 2015). Despite that flow treatment was not applied in this study, AS1842856 treatment alone led to the increase in the expression of these key lymphatic valve-forming genes, which is consistent with the inventors' previous finding that FOXO1 directly binds to the promoter region of FOXC2 to repress FOXC2 expression and FOXO1 knockdown in cultured hdLECs results in the upregulation of the valve-forming genes (Scallan et al., 2021). These data indicate that the shear stress signaling pathway is activated in the LECs with AS1842856 treatment without flow, supported by the upregulation of the major flow responsive gene KLF4. Additionally, many other valve genes including ITGA9, GJA4, and NOS3 were upregulated by AS1842856 treatment. Since it has been reported that GJA4 (Connexin37) expression is regulated by Foxc2 (Sabine et al., 2012), the upregulation of GJA4 in AS1842856 treatment could be due to 1) the inhibition of FOXO1 transcription activity by AS1842856 and 2) the increased expression of FOXC2. The same mechanisms can be applied on the upregulation of NOS3, which is a downstream target of FOXC2 (Sabine et al., 2012). Moreover, AS1842856 treatment did not upregulate the mRNA level of β-catenin but increased the nuclear localization and the activity of β-catenin on the protein level. In addition, the inventors used shFOXO1 to knockdown FOXO1 in cultured hdLECs and obtained the same increase of non-phospho β-catenin in shFOXO1 LECs compared to the cells treated with scramble control (data not shown).

To investigate how AS1842856 particularly increased the translocation of β-catenin into the nucleus, the inventors examined the protein levels of GSK3b, the key component of the β-catenin destruction complex, and its inactive form phospho-GSK3b in the AS1842856 treated LECs. Since results showed that non-phospho β-catenin was increased, meaning less β-catenin was degraded in the destruction complex, the inventors expected to observe increased levels of phospho-GSK3B, the inactive form of GSK3β and decreased levels of GSK3B. However, the expression level of phospho-GSK3β was deceased and the expression level of GSK3β was increased. Nevertheless, the increased nuclear β-catenin added one more explanation for the upregulation of FOXC2 expression because β-catenin directly binds to the promoter region of FOXC2 gene to activate FOXC2 expression. In summary, AS1842856 treatment potentially triggered the activation of a signaling cascade that regulates flow response and lymphatic valve formation without the presence of flow.

### AS1842856 Treatment and Nuclear β-Catenin Overactivation Restore the Valve Loss in Foxc2 Heterozygous Mice

Foxc2 heterozygous mice have been characterized as the mouse model for human lymphedema distichiasis disease (Kriederman et al., 2003). These mice on the C57B6 background lack ˜40-50% valves in number compared to wildtype mice, and the remaining valves have functional defects with leaky valve leaflets that cannot seal against the pressure from lymph (Scallan et al., 2021, Castorena-Gonzalez et al., 2020). Previously, the inventors showed that deletion of Foxo1 rescues both the valve loss and the valve functional defects by inducing the growth of healthy new valves in Foxc2 heterozygous mice (Scallan et al., 2021). Therefore, inventors wanted to investigate the effect of a pharmacological method in inducing valve growth in this study. The data showed AS1842856 treatment significantly increased valve growth in Foxc2 heterozygous mice. Unlike genetic deletion of Foxo1, AS1842856 treatment neither rescued the valve loss in Foxc2 heterozygous mice to the wildtype level nor increased the valve number in wildtype mice. This may be due to the low efficiency of transient Foxo1 inhibition by AS1842856 in vivo through i.p. injection, which is supported by the inventors' previous data that one allele deletion of Foxo1 did not rescue the valve loss in Foxc2 heterozygous mice (Scallan et al., 2021). Thus, these data indicate that the effect of Foxo1 inhibition is dose dependent and a threshold amount of inhibition is required to induce valve formation.

The inventors showed that increased nuclear β-catenin activity completely rescued the valve loss in the Foxc2 heterozygous mice. However, this does not mean more β-catenin is better. Since the exon 3 of the β-catenin gene is deleted by Cre recombinase, an excessive amount of β-catenin is accumulated in the cells because β-catenin can no longer be degraded in the destruction complex. The inventors previously administrated 3 injections of tamoxifen (TM) at P1, P3, and P5 in the Foxc2^{+/CreERT2} mice to completely delete Foxo1, but the same protocol cannot be used in this study. Here, the inventors discovered that 3 injections of TM induced severe vessel dilation due to the excessive amount of β-catenin (data not shown). Thus, the amount of TM was reduced with 1 injection at P1. The data showed that the mesenteric lymphatic vessels were no longer dilated, and valves were still induced by the sufficient amount of β-catenin from exon 3 deletion. Therefore, in addition to Foxo1 deletion, moderately increased β-catenin activity can rescue the valve loss in Foxc2 heterozygous mice, the mouse model for lymphedema-distichiasis.

Patients of primary lymphedema are susceptible to developing other comorbidities in addition to poor quality of life, such as, metabolic disorders and infections, which could be caused due to dysregulated Foxo1 signaling. Elevated levels of Foxo1 have long been implicated in the pathogenesis of metabolic disorders and drug resistance in certain types of cancers and small molecule inhibitors of Foxo1 have been proposed as a form of pharmacologic treatment (Pandey et al., 2016, Goto et al., 2008). Hence, drug induced inhibition of Foxo1 can be clinically relevant in the treatment of lymphedema caused by valve defects. In summary, this study helped to better understand how signaling pathways control lymphatic valve formation and how to fine tune these pathways to regulate valve formation under disease conditions.

### Methods

### Animals

All mouse husbandry and experiments were performed following the guidelines of the University of South Florida (USF) Institution of Animal Care and Use Committee (IACUC). Male and female mice were used for all experiments and all mouse models were maintained on C57BL/6J background and obtained through material transfer agreement (MTA). The Prox1-GFP mouse line was from Dr. Young Kwon Hong from University of South California (USC), LA. The Foxc2^{+/CreERT2} mouse line was obtained from Dr. Sathish Srinivasan at the Oklahoma medical research foundation. The Ctnnb1^{lox(ex3)} mice were procured with Dr. Makoto Taketo from Kyoto University, Tokyo. The Foxo1 inhibitor, AS1842856, was obtained from EMD Millipore Corporation, USA and in vivo inhibition was achieved by intraperitoneal injections (i.p.) of AS1842856 (8 mg/kg) in wildtype and Foxc2^{+/CreERT2} pups every other day from P7 to P21. For Ctnnb1 overactivation experiments, tamoxifen (TM) was injected subcutaneously into P1 Ctnnb1^{lox(ex3)}; Prox1-GFP, Foxc2^{+/CreERT2}; Prox1-GFP, and Foxc2^{+/CreERT2}; Ctnnb1^{lox(ex3)}; Prox1-GFP pups.

### Ex Vivo Analysis of Collecting Lymphatics and Lymphatic Valve Counting

TM administered postnatal pups were euthanized using an IACUC approved method and the colleting lymphatics were analyzed at the experimental timepoints. Since the small intestine contains the majority of the lymphatic vasculature in the mesentery, collecting lymphatics from the small intestine right before the colon were imaged in situ under a Zeiss V16 fluorescent microscope and the GFP images were analyzed using Fiji (ImageJ). Each lymphatic branch in the small intestine has a defined collecting lymphatic vessel that is connected to the lymph nodes as well as the pre-collecting lymphatic branches formed next to the gut wall. The segmented line tool in Fiji was used to measure the length of the collecting lymphatic appearing from the lymph node all the way to the branched pre-collecting lymphatics near the gut wall. The multi-point tool in Fiji was used to count the GFP^{high} valves on the length of the measured collecting vessel. At least 4 controls and 4 KO animals were used for every experiment.

### Whole-Mount Immunostaining

Tissues were collected and fixed in 2% paraformaldehyde (PFA) overnight on an orbital shaker (Belly Dancer, IBI Scientific) in 4° C. Next day, the fixed tissues were washed with phosphate buffered saline (PBS) three times for 10 minutes each time followed by permeabilization in 1×PBS+0.03% Triton (X-100) (PBST) for 1 hour. The tissues were blocked using 3% donkey serum in 1×PBST for 2 hours and then incubated overnight with primary antibodies in 1×PBST on an orbital shaker at 4° C. The following day, the tissues were washed with 1×PBST (4 times for 10 mins each) to remove primary antibodies and then incubated with secondary antibodies in 1×PBST for 90 minutes at room temperature on an orbital shaker (Belly button, IBI Scientific) after which the tissues were washed with 1×PBST (4 times, 10 mins each) and incubated with conjugate antibodies in PBST for 60 minutes at RT. The tissues were then washed with 1×PBST (4 times, 10 mins each) to remove secondary antibodies and treated with DAPI (Invitrogen) in PBS for 10 mins on an orbital shaker at 4° C. for nuclear visualization. The DAPI was washed with PBS (5 mins) and the tissues were mounted with ProLong Diamond Antifade Mountant (Invitrogen) onto glass slides (Superfrost Plus Microscope slides, Fisherbrand) and stored in 4° C. until further use. The slides were imaged using the Leica SP8 confocal microscope and the figures were created using Adobe Photoshop.

### Cell Culture and AS Treatment

Human dermal lymphatic endothelial cells (hdLECs) purchased from Promocell (C-12216) were cultured in EBM-V2 (PromoCell, C-22121) media on fibronectin (20 μl/well) coated 6-well plates. hdLECs were treated with 4 different conditions: DMSO (1 μM), AS1842856 (EMD Millipore Corporation) 0.1 μM, 0.5 μM and 1 μM for 3 different time points: 24, 48, and 72 hours for qRT-PCR. For western blot, hdLECs were treated with 0.5 μM AS1842856 for 48 hours.

RNA Isolation and qRT-PCR

The RNeasy Plus Mini Kit (Qiagen) was used to isolate total RNA from hdLECs per the manufacturers' instructions. cDNA synthesis was performed using the AdvantageR RT-for-PCR kit (Takara) according to the manufacturer's guidelines. qPCR was performed in a QuantStudio 6 realtime PCR system (Applied Biosystems) with TaqmanR probes (Applied Biosystems, Thermofisher) and the Cq value of each gene was normalized to the that of GAPDH.

### Cell Lysis and Western Blot

hdLECs were washed 3 times with 1×PBS and 1×RIPA buffer and 100× phosphatase inhibitor (Pierce™, Thermoscientific) was added. A cell scraper was used to scrape the hdLECs off the 6-well plate. hdLECs were pipetted and submerged in lysis RIPA solution. The lysate was collected in a 1.5 mL protein LoBind tube and incubated for 15 mins in ice on an orbital shaker (Belly button, IBI Scientific) for complete lysis. The lysates were then centrifuged for 20 mins at 18300 RPM at 4° C. to and the supernatant was collected in a separate Protein LoBind tube without disturbing the pellet. A Bicinchoninic acid assay (BCA assay, Pierce™, Thermofisher) was performed to calculate protein concentration according to manufacturer's instructions and 25 μg of the total protein was utilized to perform the western blot. The Invitrogen™ mini gel tank apparatus was used to run the western blot on a 4-12% Blot™ Bis Tris Plus polyacrylamide gel. Protein transfer to a PVDF membrane was carried out using an iBlot 2 dry blotting system (Lifesciences, Thermofisher) under the P0 module. The iBind Flex western system (Thermo Fisher Scientific) was used to process the mini blots utilizing an incubation time of >2.5 hours after which the blots were imaged using a Bio-Rad ChemiDoc imaging system.

### Western Blot Quantification

Quantification analysis of western blot bands was performed in the following manner: 1. Unsaturated western blot images (.scn) were opened with Image Lab 6.1 (Bio-Rad); 2. Using the “lane and bands” tool on the left sidebar of Image Lab, the lanes were defined and adjusted manually; 3. Using the “lane and bands” tool, the bands were identified manually with “add” bands option; 4. The “lane profile” tool was used to omit background; 5. The standard and sample lanes were labeled on the “image info” tool; 6. The adjusted volume (Int) value was obtained using the “lane analysis” tool on Image Lab; 7. Each protein's band volume was normalized to the band volume of the housekeeping protein (b-Actin); 8. Relative expression of each protein was calculated by normalizing the band volume of the experimental group (AS 0.5) to its control (DMSO).

### Statistics

Unpaired two-sided Student's t-test was used to determine significant differences between two groups. One-way ANOVA with Tukey's multiple comparisons test was used to determine significant differences for data with one independent variable. Two-way ANOVA with Tukey's multiple comparisons test were performed to determine significant differences for data with two independent variables. p<0.05 was considered significant. All data are represented as means±SEM. Graphpad Prism software (version 6) was used for all statistical analyses and to plot quantitative data.

### REFERENCES CITED

BAZIGOU, E., XIE, S., CHEN, C., WESTON, A., MIURA, N., SOROKIN, L., ADAMS, R., MURO, A. F., SHEPPARD, D. & MAKINEN, T. 2009. Integrin-alpha9 is required for fibronectin matrix assembly during lymphatic valve morphogenesis. Dev Cell, 17, 175-86.
BRESLIN, J. W., YANG, Y., SCALLAN, J. P., SWEAT, R. S., ADDERLEY, S. P. & MURFEE, W. L. 2018. Lymphatic Vessel Network Structure and Physiology. Compr Physiol, 9, 207-299.
CASTORENA-GONZALEZ, J. A., SRINIVASAN, R. S., KING, P. D., SIMON, A. M. & DAVIS, M. J. 2020. Simplified method to quantify valve back-leak uncovers severe mesenteric lymphatic valve dysfunction in mice deficient in connexins 43 and 37. J Physiol, 598, 2297-2310.
CHA, B., GENG, X., MAHAMUD, M. R., FU, J., MUKHERJEE, A., KIM, Y., JHO, E. H., KIM, T. H., KAHN, M. L., XIA, L., DIXON, J. B., CHEN, H. & SRINIVASAN, R. S. 2016. Mechanotransduction activates canonical Wnt/beta-catenin signaling to promote lymphatic vascular patterning and the development of lymphatic and lymphovenous valves. Genes Dev, 30, 1454-69.
CHOI, I., CHUNG, H. K., RAMU, S., LEE, H. N., KIM, K. E., LEE, S., YOO, J., CHOI, D., LEE, Y. S., AGUILAR, B. & HONG, Y. K. 2011. Visualization of lymphatic vessels by Prox1-promoter directed GFP reporter in a bacterial artificial chromosome-based transgenic mouse. Blood, 117, 362-5.
DAVIS, M. J., RAHBAR, E., GASHEV, A. A., ZAWIEJA, D. C. & MOORE, J. E., JR. 2011. Determinants of valve gating in collecting lymphatic vessels from rat mesentery. Am J Physiol Heart Circ Physiol, 301, H48-60.
GENG, X., CHA, B., MAHAMUD, M. R., LIM, K. C., SILASI-MANSAT, R., UDDIN, M. K., MIURA, N., XIA, L., SIMON, A. M., ENGEL, J. D., CHEN, H., LUPU, F. & SRINIVASAN, R. S. 2016. Multiple mouse models of primary lymphedema exhibit distinct defects in lymphovenous valve development. Dev Biol, 409, 218-33.
GOTO, T., TAKANO, M., HIRATA, J. & TSUDA, H. 2008. The involvement of FOXO1 in cytotoxic stress and drug-resistance induced by paclitaxel in ovarian cancers. Br J Cancer, 98, 1068-75.
HARADA, N., TAMAI, Y., ISHIKAWA, T., SAUER, B., TAKAKU, K., OSHIMA, M. & TAKETO, M. M. 1999. Intestinal polyposis in mice with a dominant stable mutation of the beta-catenin gene. EMBO J, 18, 5931-42.
JEON, H. H., YU, Q., LU, Y., SPENCER, E., LU, C., MILOVANOVA, T., YANG, Y., ZHANG, C., STEPANCHENKO, O., VAFA, R. P., COELHO, P. G. & GRAVES, D. T. 2018. FOXO1 regulates VEGFA expression and promotes angiogenesis in healing wounds. J Pathol, 245, 258-264.
JEON, H. H., YU, Q., WITEK, L., LU, Y., ZHANG, T., STEPANCHENKO, O., SON, V. J., SPENCER, E., OSHILAJA, T., SHIN, M. K., ALAWI, F., COELHO, P. G. & GRAVES, D. T. 2021. Clinical application of a FOXO1 inhibitor improves connective tissue healing in a diabetic minipig model. Am J Transl Res, 13, 781-791.
KANADY, J. D., DELLINGER, M. T., MUNGER, S. J., WITTE, M. H. & SIMON, A. M. 2011. Connexin37 and Connexin43 deficiencies in mice disrupt lymphatic valve development and result in lymphatic disorders including lymphedema and chylothorax. Dev Biol, 354, 253-66.
KANADY, J. D., MUNGER, S. J., WITTE, M. H. & 532 SIMON, A. M. 2015. Combining Foxc2 and Connexin37 deletions in mice leads to severe defects in lymphatic vascular growth and remodeling. Dev Biol, 405, 33-46.
KAZENWADEL, J., BETTERMAN, K. L., CHONG, C. E., STOKES, P. H., LEE, Y. K., SECKER, G. A., AGALAROV, Y., DEMIR, C. S., LAWRENCE, D. M., SUTTON, D. L., TABRUYN, S. P., MIURA, N., SALMINEN, M., PETROVA, T. V., MATTHEWS, J. M., HAHN, C. N., SCOTT, H. S. & HARVEY, N. L. 2015. GATA2 is required for lymphatic vessel valve development and maintenance. J Clin Invest, 125, 2979-94.
KAZENWADEL, J., SECKER, G. A., LIU, Y. J., ROSENFELD, J. A., WILDIN, R. S., CUELLAR RODRIGUEZ, J., HSU, A. P., DYACK, S., FERNANDEZ, C. V., CHONG, C. E., BABIC, M., BARDY, P. G., SHIMAMURA, A., ZHANG, M. Y., WALSH, T., HOLLAND, S. M., HICKSTEIN, D. D., HORWITZ, M. S., HAHN, C. N., SCOTT, H. S. & HARVEY, N. L. 2012. Loss-of-function germline GATA2 mutations in patients with MDS/AML or MonoMAC syndrome and primary lymphedema reveal a key role for GATA2 in the lymphatic vasculature. Blood, 119, 1283-91.
KRIEDERMAN, B. M., MYLOYDE, T. L., WITTE, M. H., DAGENAIS, S. L., WITTE, C. L., RENNELS, M., BERNAS, M. J., LYNCH, M. T., ERICKSON, R. P., CAULDER, M. S., MIURA, N., JACKSON, D., BROOKS, B. P. & GLOVER, T. W. 2003. FOXC2 haploinsufficient mice are a model for human autosomal dominant lymphedema-distichiasis syndrome. Hum Mol Genet, 12, 1179-85.
NAGASHIMA, T., SHIGEMATSU, N., MARUKI, R., URANO, Y., TANAKA, H., SHIMAYA, A., SHIMOKAWA, T. & SHIBASAKI, M. 2010. Discovery of novel forkhead box 01 inhibitors for treating type 2 diabetes: improvement of fasting glycemia in diabetic db/db mice. Mol Pharmacol, 78, 961-70.
NORRMEN, C., IVANOV, K. I., CHENG, J., ZANGGER, N., DELORENZI, M., JAQUET, M., MIURA, N., PUOLAKKAINEN, P., HORSLEY, V., HU, J., AUGUSTIN, H. G., YLA HERTTUALA, S., ALITALO, K. & PETROVA, T. V. 2009. FOXC2 controls formation and maturation of lymphatic collecting vessels through cooperation with NFATc1. J Cell Biol, 185, 439-57.
OLIVER, G. 2004. Lymphatic vasculature development. Nat Rev Immunol, 4, 35-45.
OSTERGAARD, P., SIMPSON, M. A., CONNELL, F. C., STEWARD, C. G., BRICE, G., WOOLLARD, W. J., DAFOU, D., KILO, T., SMITHSON, S., LUNT, P., MURDAY, V. A., HODGSON, S., KEENAN, R., PILZ, D. T., MARTINEZ-CORRAL, I., MAKINEN, T., MORTIMER, P. S., JEFFERY, S., TREMBATH, R. C. & MANSOUR, S. 2011. Mutations in GATA2 cause primary lymphedema associated with a predisposition to acute myeloid leukemia (Emberger syndrome). Nat Genet, 43, 929-31.
PANDEY, A., KUMAR, G. S., KADAKOL, A., MALEK, V. & GAIKWAD, A. B. 2016. FoxO1 Inhibitors: The Future Medicine for Metabolic Disorders? Curr Diabetes Rev, 12, 223-30.
PETROVA, T. V., KARPANEN, T., NORRMEN, C., MELLOR, R., TAMAKOSHI, T., FINEGOLD, D., FERRELL, R., KERJASCHKI, D., MORTIMER, P., YLA-HERTTUALA, S., MIURA, N. & ALITALO, K. 2004. Defective valves and abnormal mural cell recruitment underlie lymphatic vascular failure in lymphedema distichiasis. Nat Med, 10, 974-81.
SABINE, A., AGALAROV, Y., MABY-EL HAJJAMI, H., JAQUET, M., HAGERLING, R., POLLMANN, C., BEBBER, D., PFENNIGER, A., MIURA, N., DORMOND, O., CALMES, J. M., ADAMS, R. H., MAKINEN, T., KIEFER, F., KWAK, 576 B. R. & PETROVA, T. V. 2012. Mechanotransduction, PROX1, and FOXC2 cooperate to control connexin37 and calcineurin during lymphatic-valve formation. Dev Cell, 22, 430-45.
SABINE, A., BOVAY, E., DEMIR, C. S., KIMURA, W., JAQUET, M., AGALAROV, Y., ZANGGER, N., SCALLAN, J. P., GRABER, W., GULPINAR, E., KWAK, B. R., MAKINEN, T., MARTINEZ-CORRAL, I., ORTEGA, S., DELORENZI, M., KIEFER, F., DAVIS, M. J., DJONOV, V., MIURA, N. & PETROVA, T. V. 2015. FOXC2 and fluid shear stress stabilize postnatal lymphatic vasculature. J Clin Invest, 125, 3861-77.
SCALLAN, J. P., KNAUER, L. A., HOU, H., CASTORENA-GONZALEZ, J. A., DAVIS, M. J. & YANG, Y. 2021. Foxo1 deletion promotes the growth of new lymphatic valves. J Clin Invest, 131.
SWEET, D. T., JIMENEZ, J. M., CHANG, J., HESS, P. R., MERICKO-ISHIZUKA, P., FU, J., XIA, L., DAVIES, P. F. & KAHN, M. L. 2015. Lymph flow regulates collecting lymphatic vessel maturation in vivo. J Clin Invest, 125, 2995-3007.
WANG, D., WANG, Y., ZOU, X., SHI, Y., LIU, Q., HUYAN, T., SU, J., WANG, Q., ZHANG, F., LI, X. & TIE, L. 2020. FOXO1 inhibition prevents renal ischemia-reperfusion injury via cAMP-response element binding protein/PPAR-gamma coactivator-1alpha-mediated mitochondrial biogenesis. Br J Pharmacol, 177, 432-448.
YANG, Y., CHA, B., MOTAWE, Z. Y., SRINIVASAN, R. S. & SCALLAN, J. P. 2019. VE-Cadherin Is Required for Lymphatic Valve Formation and Maintenance. Cell Rep, 28, 2397-2412 e4.
YANG, Y. & OLIVER, G. 2014. Development of the mammalian lymphatic vasculature. J Clin Invest, 124, 888-97.
ZOU, P., LIU, L., ZHENG, L., LIU, L., STONEMAN, R. E., CHO, A., EMERY, A., GILBERT, E. R. & CHENG, Z. 2014. Targeting FoxO1 with AS1842856 suppresses adipogenesis. Cell Cycle, 13, 3759-67.

1. A method for treating congenital lymphedema in a subject in need thereof, the method comprising administering to the subject a therapeutically effective amount of a FOXO1 inhibitor.

2. The method of claim 1, wherein the subject has lymphedema-distichiasis syndrome.

3. The method of claim 1, wherein the subject is between about 0 and about 60 months old.

4. The method of claim 1, wherein the FOXO1 inhibitor is administered intraperitoneally.

5. The method of claim 1, wherein the FOXO1 inhibitor is AS1842856.

6. The method of claim 5, wherein the AS1842856 is administered at a dose of between about 0.05 mg/kg and about 10 mg/kg.

7. The method of claim 6, wherein the AS1842856 is administered at a dose of about 8 mg/kg.

8. The method of claim 7, wherein the AS1842856 is administered at about every other day.

9. The method of claim 1, wherein the subject is a human or a non-human mammal.

10. A method for increasing lymphatic valve growth in a subject in need thereof, the method comprising administering to the subject a FOXO1 inhibitor.

11. The method of claim 10, wherein the FOXO1 inhibitor is administered intraperitoneally.

12. The method of claim 10, wherein the FOXO1 inhibitor is AS1842856.

13. The method of claim 10, wherein the subject is a human or a non-human mammal.

14. A method for increasing nuclear β-catenin activity in a cell, the method comprising contacting the cell with a FOXO1 inhibitor.

15. The method of claim 14, wherein the cell is a lymphatic endothelial cell.

16. The method of claim 15, wherein the cell is a human dermal lymphatic endothelial cell.

17. The method of claim 14, wherein the FOXO1 inhibitor is AS1842856.

18. The method of claim 17, wherein the cell is contacted with the AS1842856 for about 48 hours.

19. The method of claim 18, wherein the cell is contacted with the AS1842856 at a concentration of between about 0.1 μM and about 1 μM.

20. The method of claim 19, wherein the cell is contacted with the AS1842856 at a concentration of about 0.5 μM.
